(19) United States
(12) Patent Application Publication
(10) Pub. No.: US 2022/0008761 A1
(43) Pub. Date: Jan. 13, 2022

(54) SMART MASKS

(71) Applicant: XUPERMASK OPCO, LLC, Los Angeles, CA (US)

(72) Inventor: William Adams, Los Angeles, CA (US)

(21) Appl. No.: 17/300,299

(22) Filed: Jul. 10, 2020

(51) Int. Cl. A62B 18/02; A62B 18/08; A62B 19/00

(52) U.S. Cl. CPC A62B 18/025; A61L 9/20; A62B 19/00; A62B 18/08

(57) ABSTRACT

A smart mask includes a face-covering, an attachment element, one or more power source(s), an air circulation system, an exhaust system, and a controller. The face-covering is configured to cover a face area of a wearer. The attachment element is configured to attach the face-covering onto the face area of the wearer. The air circulation system is powered by at least one of the one or more power source(s) and configured to filter outside air and draw the filtered outside air into the face area. The exhaust system is placed below the air circulation system and configured to purge exhaled air out of the face area. The controller is configured to control the air circulation system.

### BACKGROUND

Protective respirators or masks are pieces of fabric, kits, or equipment worn on the head and face to protect the wearer from inhaling hazardous atmospheres, including fumes, vapors, gases, or particulate matter such as dust and airborne microorganisms. Protective respirators or masks come in many different configurations and ratings. The primary ratings are N, P, and R. Following the letter rating is a number, generally, 95, 99, or 100, which relates to how much the filter has been measured to remove of particulate matter of 0.3 microns in diameter or greater. “N” represents “Not oil resistant”, “R” represents “Resistant to oil”, and “P” represents “Oil Proof”. “95” indicates that the mask removes 95% of all particles that are at least 0.3 microns in diameter, “99” indicates that the mask removes 99% of all particles that are at least 0.3 microns in diameter, and “100” represents that the mask removes 99.97% of all particles that are at least 0.3 microns in diameter.

In the past, such protective masks are mostly worn by professionals (e.g., healthcare professionals and construction professionals). However, since the COVID-19 was declared a pandemic, many countries required their citizens to wear masks while in public during the pandemic. A growing number of U.S. states have also mandated the use of masks and face coverings while in public during the pandemic. Fabric masks and disposable paper masks are the most popular. However, many of these masks either do not have sufficient breathability or cannot provide sufficient protection. Wearing such a mask can also interfere with a user's capability of listening to music, answering phone calls, etc.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter.

The embodiments described herein are related to smart devices and protective respirators or masks (hereinafter referred to as smart masks) that are configured to protect a wearer from inhaling hazardous atmospheres.

The smart mask includes a face-covering configured to cover a face area of a wearer and an attachment element configured to attach the face-covering onto the face area of the wearer. The smart mask also includes one or more power source(s), an air circulation system, and an exhaust system. The air circulation system is powered by at least one of the one or more power source(s) and configured to filter outside air and draw the filtered outside air into the face area. The exhaust system is placed below the air circulation system and configured to purge exhaled air out of the face area. The smart mask also includes a controller configured to control the air circulation system.

In some embodiments, the air circulation system includes one or more filter receptacle(s), one or more fan(s), and one or more puck(s). Each of the one or more filter receptacle(s) is configured to receive a filter cartridge for filtering the outside air drawn into the face area. Each of the one or more fan(s) is powered by at least one of the one or more power source(s) and configured to draw outside air through the corresponding filter cartridge into the face area. Each of the puck(s) is configured to cover the corresponding filter cartridge or fan. In some embodiments, each fan is configured to operate at different speeds (e.g., low, medium, high, etc.). The controller is configured to control a speed of each fan.

In some embodiments, the one or more fan(s) include a left fan placed on a left side of the face-covering and a right fan placed on a right side of the face-covering. The one or more filter receptacle(s) includes a left filter receptacle and a right filter receptacle. The left receptacle is coupled to the left fan and configured to receive a left filter cartridge, and the right receptacle is coupled to the right fan and configured to receive a right filter cartridge. The one or more puck(s) includes a left puck and a right puck. The left puck is configured to cover the left fan and the left filter cartridge, and the right puck is configured to cover the right fan and the right filter cartridge.

In some embodiments, each filter receptacle includes a magnetic portion configured to hold a metal frame of the corresponding filter cartridge in place magnetically. In some embodiments, each filter receptacle has a circular-shaped recess configured to receive a circular-shaped filter cartridge.

In some embodiments, the smart mask further includes one or more ultraviolet light sources (e.g., ultraviolet LEDs). Each ultraviolet light source is powered by at least one of the one or more power source(s) and configured to disinfect the face covering and/or an area around each filter cartridge. The controller is further configured to control the one or more ultraviolet light source(s). In some embodiments, each ultraviolet light source is coupled to a corresponding puck or a corresponding filter receptacle. In some embodiments, one or more ultraviolet light(s) is positioned around each filter cartridge. In some embodiments, each ultraviolet light source is configured to emit ultraviolet light at different intensities. The controller is further configured to control an intensity of each ultraviolet light source.

In some embodiments, the smart mask further includes an audio system. The audio system is powered by at least one of the one or more power source(s) and configured to receive a sound signal wirelessly from a user terminal. The controller is further configured to control the audio system. In some embodiments, the audio system includes at least a pair of earbuds, or a pair of speakers. In some embodiments, each of the pair of earbuds includes a magnetic portion or a metal portion, and the attachment element also includes a pair of magnetic portion or metal portions corresponding to the pair of earbuds, such that when the pair of earbuds are not in use, each earbud is configured to be secured to the corresponding magnetic portion or metal portion of the attachment element magnetically.

In some embodiments, the audio system also includes a microphone configured to receive a voice input. The controller is further configured to process the voice input to identify one or more voice command(s) for controlling the air circulation system or the audio system.

In some embodiments, the smart mask further includes a network interface configured to connect to a user terminal or a computer network. When the user terminal is connected to the network interface, the network interface is configured to transmit status data to the user terminal or a cloud storage, and/or to grant the user terminal control of the air circulation system.

In some embodiments, the smart mask further includes a temperature sensor configured to detect a body temperature of the wearer. In yet some other embodiments, the smart mask further includes a heart rate monitor configured to detect a heart rate of the wearer.

In some embodiments, the one or more power source(s) include one or more rechargeable batteries, and the smart mask further includes a charging port (e.g., a USBC port, a micro USB port, etc.) configured to charge the one or more rechargeable batteries.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and details through the use of the accompanying drawings in which:

FIG. 1A illustrates a front view of an example smart mask being worn by a wearer;

FIG. 1B illustrates a front view of the example smart mask not being worn;

FIG. 1C illustrates a right-side view of the example smart mask being worn by a wearer;

FIG. 1D illustrates a left-side view of the example smart mask being worn by a wearer;

FIG. 1E illustrates a right-side view of the example smart mask not being worn;

FIG. 1F illustrates a left-side view of the example smart mask not being worn;

FIG. 1G illustrates an example face-covering, on which two pucks have been removed;

FIG. 1H illustrates an example filter cartridge;

FIG. 1I illustrates an example fan and an example filter cartridge that fits on top of the fan;

FIGS. 1J and 1K illustrate example controllers that are placed on the left or right side of the face-covering;

FIG. 2 illustrates an example architecture of the smart mask;

FIG. 3 illustrates an example control interfaces that may allow a wearer to control or interface with the smart mask;

FIG. 4A illustrates an example home page a mobile application for controlling and interfacing with the smart mask;

FIG. 4B illustrates an example air control interface of the mobile application for controlling an air circulation system of the smart mask; and

FIG. 5 illustrates an example computing system in which the principles described herein may be employed.

### DETAILED DESCRIPTION

The embodiments described herein are related to smart devices and protective respirators or masks (hereinafter referred to as smart masks) that are configured to protect a wearer from inhaling hazardous atmospheres.

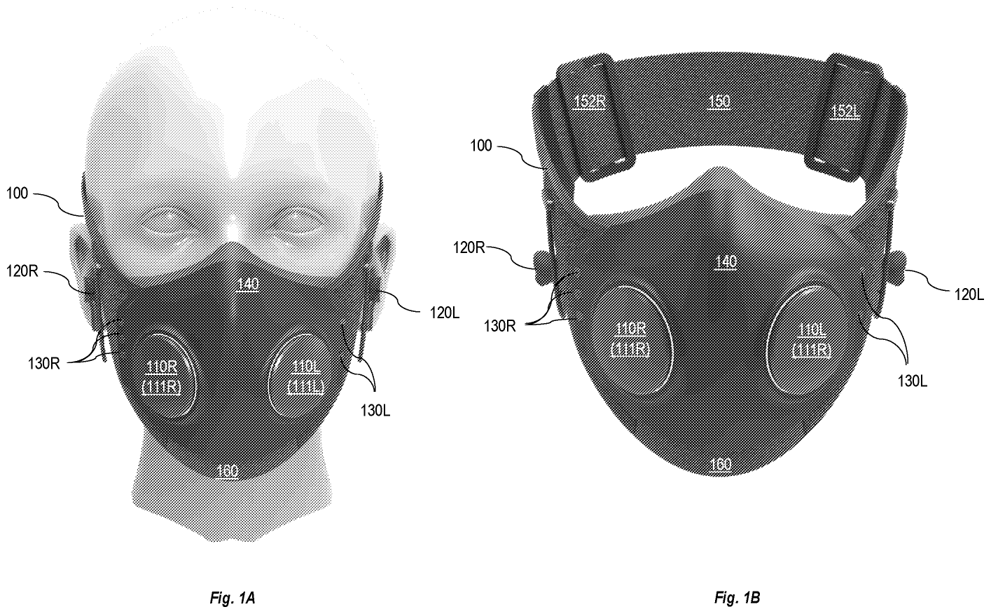

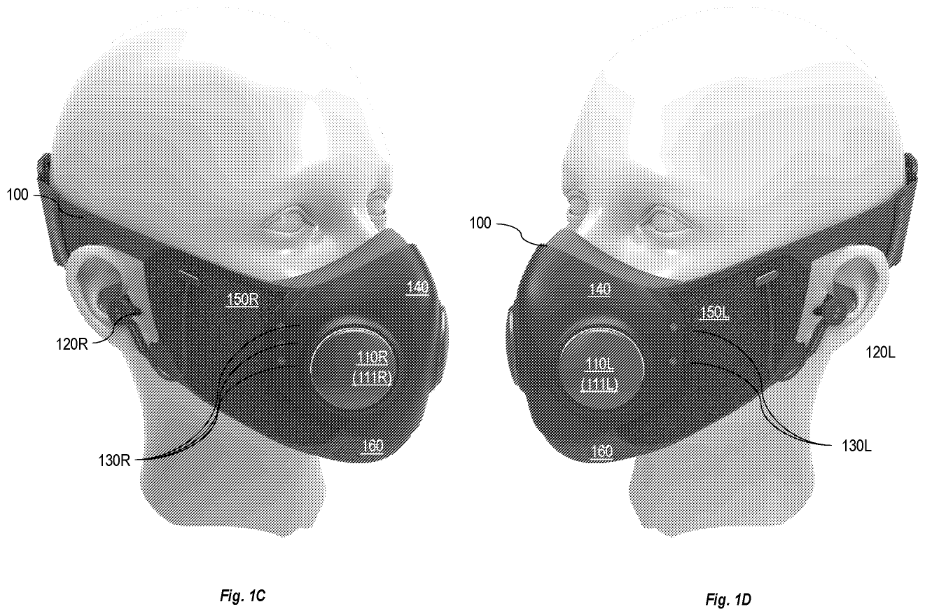

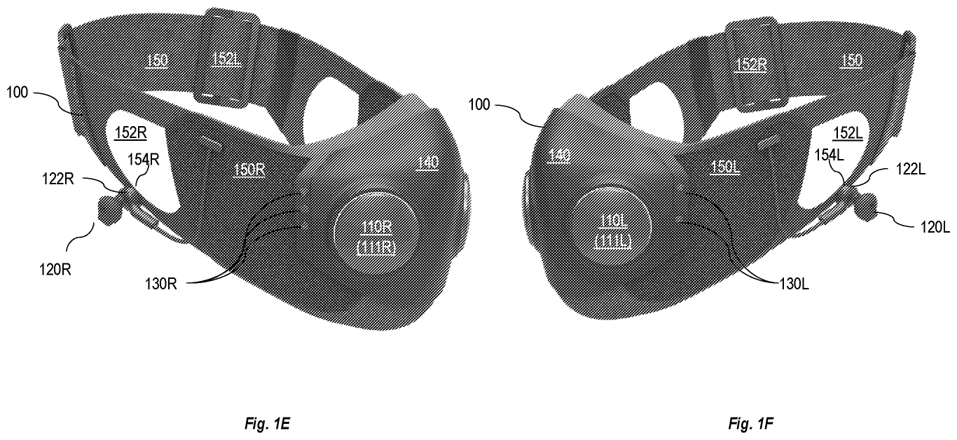

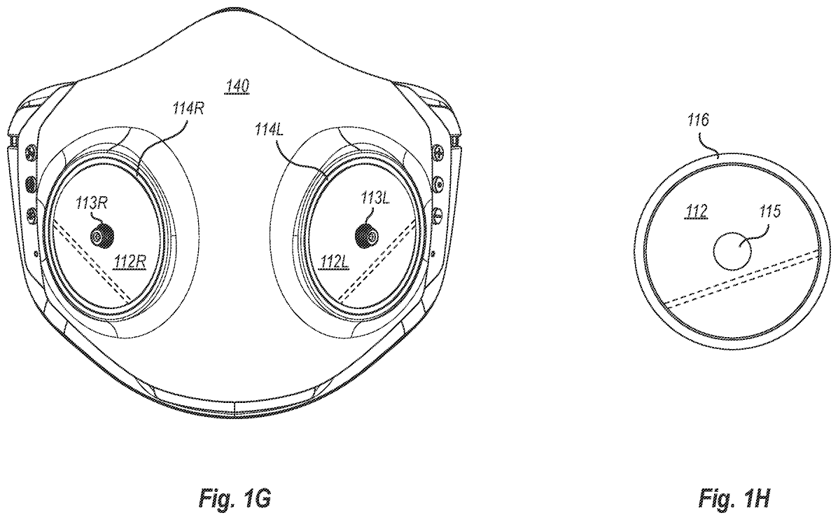

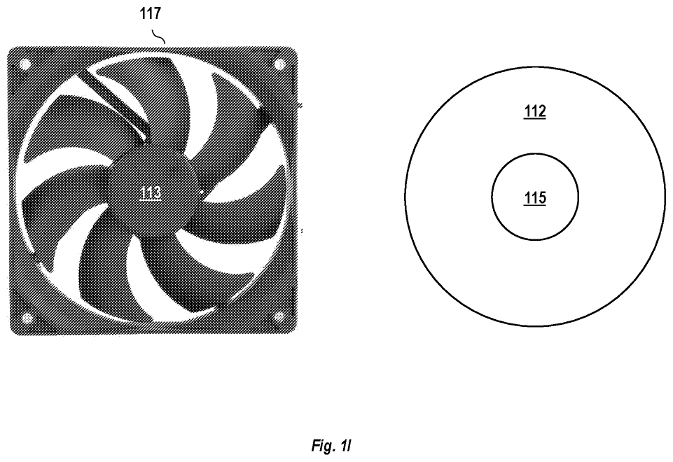

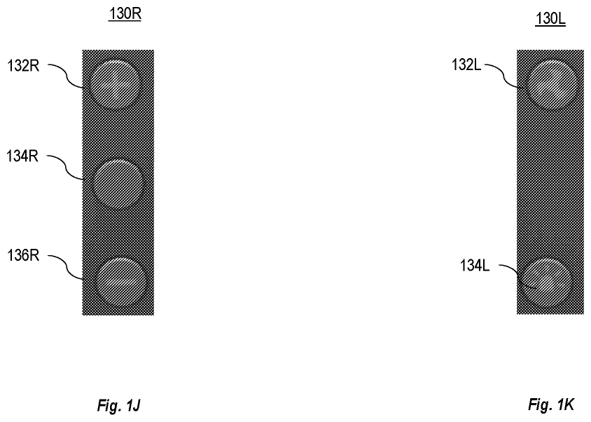

FIGS. 1A through 1F illustrate an example embodiment of the smart mask 100. FIGS. 1A and 1B illustrate a front view of the smart mask 100 when the smart mask 100 is worn or not worn by a wearer. FIGS. 1C and 1D illustrate a right-side view and a left side view of the smart mask 100, when the smart mask 100 is worn by a wearer. FIGS. 1E and 1F illustrate a right-side view and a left side view of the smart mask 100 when the smart mask 100 is not worn.

As illustrated in FIGS. 1A through 1F, the smart mask 100 includes a face-covering 140, an attachment element 150, an air circulation system 110R, 110L, an exhaust system 160, an audio system 120R, 120L, and one or more controller(s) 130R, 130L. The face-covering 140 is configured to cover a face area of a wearer. The attachment element 150 is configured to attach the face-covering 140 onto the face area of the wearer. The air circulation system 110R, 110L is configured to filter outside air and draw the filtered outside air into the face area, such that the wearer can breathe easily, and the air in the face area is kept cool and dry. The exhaust system 160 is configured to purge exhaled are out of the face area. The controller(s) 130R, 130L are configured to control the air circulation system 110R, 110L and the audio system 120R, 120L.

The air circulation system 110R and 110L includes a right portion 110R placed on a right side of the face-covering 140, and a left portion 110L placed on a left side of the face-covering 140. The exhaust system 160 is placed below the air circulation system 110R, 110L. The audio system 120R, 120L, includes a pair of earbuds 120R and 120L. The attachment element 150 may be an adjustable band, including one or more adjustable buckles 152R and 152L configured to adjust the size of the band.

Further, referring to FIGS. 1E and 1F, in some embodiments, each of the pair of earbuds 120R, 120L further includes a magnetic portion or a metal portion 122R or 122L, and the attachment element 150 also includes a pair of magnetic portions or metal portions 154R, 154L near the pair of earbuds on the right side 150R or left side 150L, such that when the earbuds are not worn, they are configured to be secured to the corresponding magnetic portion or metal portion 154R, 154L of the attachment element 150.

In embodiments, each side of the air circulation system 110R or 110L includes a filter receptacle, a fan, and a removable puck 111R or 111L. The filter receptacle is configured to receive a filter cartridge for filtering the outside air drawn into the face area. The filter cartridge may be designed to meet various standard ratings, such as N95, N99, or N100. The fan is configured to draw outside air through the corresponding filter cartridge into the face area. The fan not only can help a wearer to breathe easily, but also prevent moisture and heat from building up in the face area. The removable puck 111R or 111L is configured to cover the corresponding filter cartridge or fan.

FIG. 1G illustrates a face-covering 140, on which the pucks 111 (including 111R and 111L) are removed. FIG. 1H illustrates an example embodiment of the filter cartridge 112. When the pucks 111 are removed, the filters 112 (112R or 112L) is exposed to a user, and the user can easily replace the filters 112 if needed. In embodiments, each filter receptacle 144 (114R or 114L) includes a circular-shaped recess configured to receive a circular-shaped filter cartridge 112. In some embodiments, each filter receptacle 114 further includes a magnetic portion, and each filter cartridge 112 also includes a metal frame 116 configured to be secured in the filter receptacle 114.

FIG. 11 illustrates an example embodiment of a fan 117. In embodiments, the filter receptacle 114 and the filter cartridge 112 are positioned on top of the fan 117. Each filter cartridge 112 may include a center aperture 115, through which a nose 113 of the corresponding fan 117 protrudes.

In some embodiments, the smart mask 100 further includes one or more light source(s). In some embodiments, the one or more light source(s) are indicators, indicating whether the fan is running. In some embodiments, the one or more light source(s) are decorations to provide a glow effect at the puck area. In yet some other embodiments, one or more light source(s) includes one or more ultraviolet light source(s) configured to emit ultraviolet light to disinfect the face-covering 140, the filter receptacle, the puck and/or an area of around the filter cartridge.

As illustrated in FIGS. 1A through 1F, the smart mask 100 includes one or more controllers 130R and 130L. In embodiments, one or more controllers (e.g., buttons) 130R are positioned on the right side of the face-covering, and one or more controllers (e.g., buttons) 130L are positioned on the left side of the face-covering 140. FIGS. 1J and 1K illustrate an example embodiment of the controllers 130R and 130L. Referring to FIG. 1J, the controller 130R is configured to control the audio system 120R, 120L. In embodiments, the controller 130R includes a volume up button 132R, a volume down button 136R, and a center button 134R. A wearer may use the volume up button 132R or volume down button 136R to control the volume of the audio system. A wearer may use the center button 134R to pause or play a song or audio file. A wearer may also use the controllers 132R, 134R, and/or 136R to fast-forward, rewind, and/or skip a song or audio file, by pressing or tapping the controllers 132R, 134R, and/or 134R once, twice, three times, and/or long-pressing, etc.

Referring to FIG. 1K, the controller 130L is configured to control the fan(s) and the light source(s). In embodiments, the controller 130L includes a fan button 132L and a light button 134L. A wearer may use the fan button 132L to turn on or off the fan(s), and use the light button 134L to turn on or off the light source(s). In some embodiments, the fan(s) may be configured to operate at different speeds (e.g., low, medium, high, etc.), and the fan button 132L may further be used to control the speed of the fan(s). In some embodiments, the light source(s) may be configured to emit a light beam at different intensities, and the light button 134L may further be used to control the intensities of the light.

In embodiments, each of the fan(s), the earbud(s), and the light source(s) is configured to operate on direct current (DC) electricity. Thus, the smart mask 100 also includes one or more DC power source(s), such as batteries. For example, each earbud may use a set of batteries that operates at about 3-4 volts DC power, and each fan may use a set of batteries that operates at about 5 volts DC power. In some embodiments, the smart mask 100 is configured to be powered by disposable batteries. In some embodiments, the smart mask 100 is configured to be powered by rechargeable batteries. In such embodiments, the smart mask 100 also includes one or more charging port(s) (e.g., USBC port, or micro USB port) for recharging the rechargeable batteries.

In addition to the above-described components, the smart mask 100 may also include one or more microphone(s), one or more sensors (such as a temperature sensor, a heart rate monitor, etc.) and one or more network interface(s) (such as Bluetooth interface, WiFi interface, NFC interface, etc.). In some embodiments, the one or more microphone(s) allows the user to make phone calls or provide voice command to a user terminal. In some embodiments, the one or more controller(s) may be configured to receive and process voice input from the microphone(s) to identify one or more voice command(s), such as “turn on/off the fan”, “turn on/off the light”, etc.

In some embodiments, the temperature sensor may be coupled to at least one of the earbuds 120 to detect an inner-ear temperature of the wearer. In some embodiments, the heart rate monitor may be coupled to the attachment element 150 to detect a heart rate of the wearer. In some embodiments, the network interface(s) is configured to connect to a user terminal (e.g., a mobile device) and/or a cloud service to transmit status data and/or grant control of the smart mask 100 to the user terminal. For example, the user terminal may be required to install a mobile app associated with the smart mask 100. After installing the mobile app, the user terminal is allowed to receive various status data and sensor data from the smart mask 100, and the user terminal is also able to control the smart mask 100 via the mobile app, such as turn on or off the air circulation system, turn on or off the light sources, and/or turn on or off the audio system.

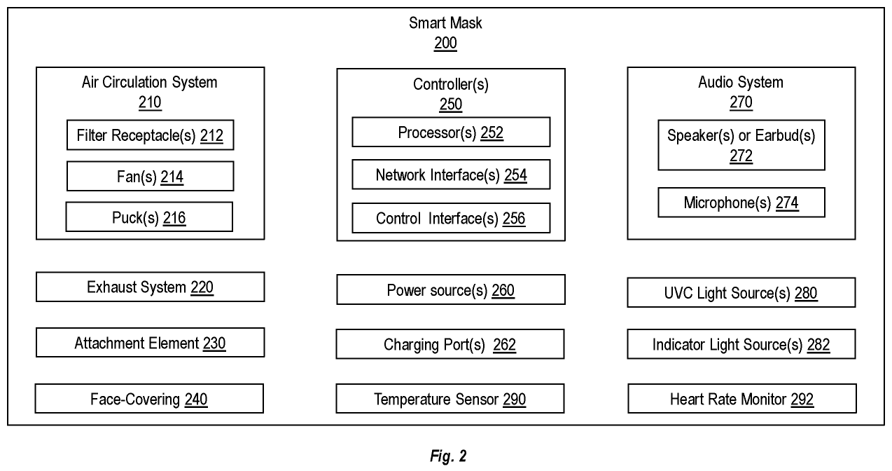

FIG. 2 illustrates an example architecture 200 of the smart mask, which corresponds to the smart mask 100 of FIGS. 1A through 1G. As illustrated in FIG. 2, the smart mask 200 includes a face-covering 240, an attachment element 230, an air circulation system 210, an exhaust system 220, one or more power source(s) 260, and one or more controller(s) 250. The face-covering 240 is configured to cover a face area of a wearer. The attachment element 230 is configured to attach the face-covering 240 onto the face area of the wearer. The air circulation system 210 is powered by at least one of the one or more power source(s) 260 and configured to filter outside air and draw the filtered outside air into the face area. The exhaust system 220 is placed below the air circulation system and configured to purge exhaled air out of the face area. The one or more controller(s) 250 is configured to control the smart mask 200, including but not limited to the air circulation system 210.

In embodiments, the air circulation system 210 includes one or more filter receptacle(s) 212, one or more fan(s) 214, and one or more puck(s) 216. Each of the one or more filter receptacle(s) 212 is configured to receive a filter cartridge for filtering the outside air drawn into the face area. Each of the one or more fan(s) 214 is powered by at least one of the one or more power source(s) 260 and configured to draw outside air through the corresponding filter cartridge into the face area. Each of the one or more puck(s) 216 is configured to removably cover the corresponding filter cartridge or fan 214.

In some embodiments, the one or more fan(s) 214 includes a left fan placed on a left side of the face area and a right fan placed on a right side of the face area. The one or more filter receptacle(s) 212 includes a left filter receptacle and a right filter receptacle. The left filter receptacle is coupled to the left fan and configured to receive a left filter cartridge, and the right filter receptacle is coupled to the right fan and configured to receive a right filter cartridge. The one or more puck(s) 216 includes a left puck and a right puck. The left puck is configured to cover the left fan and the left filter cartridge, and the right puck is configured to cover the right fan and the right filter cartridge. In some embodiments, each of the one or more filter receptacle(s) 212 includes a magnetic portion configured to hold a metal frame of a filter cartridge in place magnetically. In some embodiments, each of the one or more filter receptacle(s) 212 has a circular-shaped recess configured to receive a circular-shaped filter cartridge.

In some embodiments, each of the one or more fan(s) 214 is configured to operate at different speeds, and the controller 250 is configured to control a speed of the one or more fan(s) 214.

In some embodiments, the smart mask 200 also includes an audio system 270. The audio system 270 is powered by at least one of the one or more power source(s) 260 and configured to receive a sound signal wirelessly from a user terminal. The controller 250 is further configured to control the audio system 270. In some embodiments, the audio system 270 includes one or more speakers (e.g., a pair of speakers) or one or more earbuds (e.g., a pair of earbuds) 272. In some embodiments, the one or more speakers or earbuds 272 are Bluetooth speakers or Bluetooth earbuds that are configured to receive sound signals from a user terminal via BLE beacons. In some embodiments, each of the earbuds 272 includes a magnetic portion or a metal portion, and the attachment element 230 also includes a pair of magnetic portions or metal portions corresponding to the pair of earbuds, such that when the pair of earbuds are not in use, each of the pair of earbuds is configured to be secured to the corresponding magnetic portion or metal portion of the attachment element 230 magnetically.

In some embodiments, the audio system 270 also includes a microphone 274 that is configured to receive voice inputs. The one or more controller(s) 250 is further configured to process the voice input to identify one or more voice command(s) for controlling the smart mask 200, including (but not limited to) controlling the air circulation system or the audio system.

In some embodiments, the smart mask 200 further includes one or more ultraviolet (UV) light source(s) 280 (e.g., one or more UV LEDs) or indicator light source(s) 282 (e.g., one or more white or color LEDs). The UV light source(s) 280 and the indicator light source(s) 282 are also powered by at least one of the one or more power source(s) 260. Each of the one or more UV light source(s) 280 and/or the indicator light source(s) 282 may be coupled to a corresponding one of the one or more puck(s) 216 or a corresponding one of the one or more filter receptacle(s) 212. In some embodiments, one or more ultraviolet light(s) 280 is positioned around each filter. The UV light source(s) 280 is configured to disinfect the face-covering, the filter receptacle, the puck and/or an area of around the filter cartridge. The indicator light source(s) 282 may be configured to indicate whether the corresponding fan 214 is on or off, or merely to cause the corresponding puck 216 to glow.

In some embodiments, the smart mask 200 may also include a temperature sensor 290 and/or a heart rate monitor 292. The temperature sensor 290 may be coupled to at least one of the earbud(s) 272 to detect the inner-ear temperature of a wearer. The heart rate monitor 292 may be coupled to the attachment element 230 to detect a heart rate of the wearer.

In some embodiments, the one or more power source(s) 260 are one or more batteries. For example, each of the earbud(s) may be powered by a separate set of batteries, operating at around 3-4 volts DC power, and each of the fan(s) may be powered by a separate set of batteries, operating at around 5 volts DC power. In some embodiments, the one or more batteries are rechargeable batteries, and the smart mask 200 also includes one or more charging port(s) 262 (e.g., USBC port, micro USB port) for charging the rechargeable batteries.

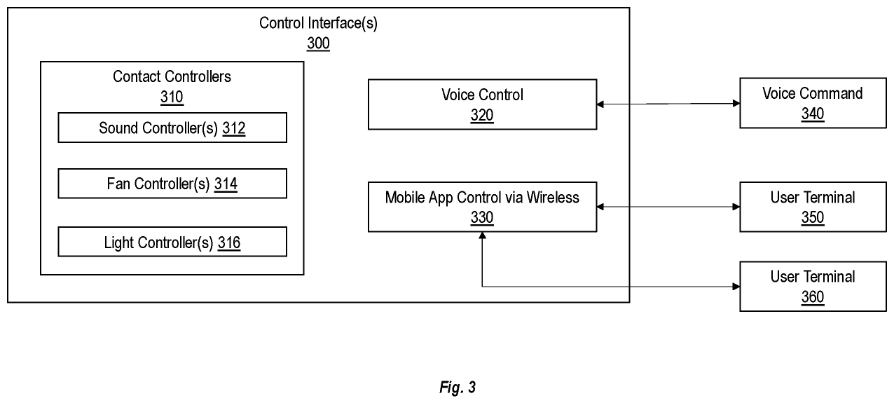

In some embodiments, some of the controller(s) 250 may be as simple as a power switch. In some embodiments, the controller(s) 250 may be a computing system that includes one or more processor(s) 252, one or more network interface(s) 254, and one or more control interface(s) 256. A control interface is an interface 256 that allows a user or wearer to interact with the various components of the smart mask 200. FIG. 3 further illustrates an example control interface(s) 300 that corresponds to the control interface(s) 256.

As illustrated in FIG. 3, the control interface(s) 300 includes contact controllers 310, which correspond to the controllers 130R and 130L illustrated in FIGS. 1A through 1F and FIGS. 1J and 1K. These contact controllers 310 may include one or more sound controller(s) 312, one or more fan controller(s) 314, and one or more light controller(s) 316. When the smart mask 100, 200 includes additional components (e.g., a microphone 274, a heart rate monitor 292, a temperature sensor 290, etc.), additional contact controllers may be implemented to control these additional components.

In some embodiments, the control interface(s) 300 may also include a voice control 320 that is configured to receive and process voice commands from a wearer. In some embodiments, the voice control 320 may simply be able to recognize a few pre-programmed commands, such as “turn on the fan”, “turn on the light”, etc. In some embodiments, the voice control 320 may include a smart AI component that is configured process natural language voice commands and perform complex tasks based on the processed natural language voice commands. The control interface(s) 300 may also use wireless interface(s) to communicate with a cloud service 360 and/or a mobile application installed on a user terminal 350 to allow a user to control the smart mask 100, 200 via the mobile application.

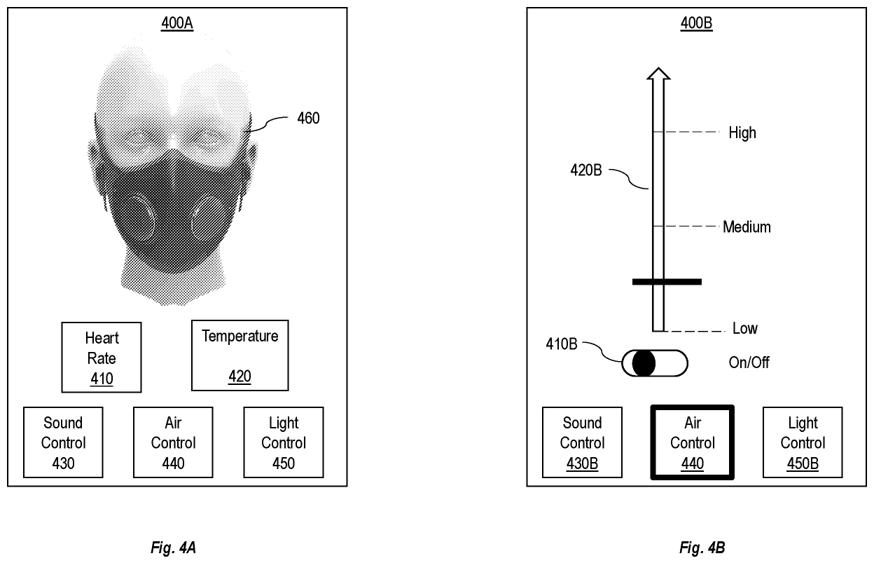

FIGS. 4A and 4B illustrate two example mobile application user interfaces 400A and 400B. FIG. 4A illustrates an example home page (or default view) 400A of the mobile application. From the home page 400A, a user can navigate to various control functions of the smart mask 100, 200. For example, the wearer can tap the heart rate icon 410 to review his/her current heart rate. Alternatively, or in addition, the heart rate monitor is configured to record the wearer's heart rate over a period, and the wearer can tap the heart rate icon 410 to review his/her heart rates over the period. Similarly, the wearer can tap the temperature icon 420 to review his/her current body temperature or body temperatures over a period. Further, the wearer may tap the sound control icon 430, the air control icon 440, or the light control icon 450 to enter a control interface for controlling the audio system, the air circulation system, and/or the light sources.

In some embodiments, an image 460 of the smart mask may also be shown in the user interface 400, and the wearer may touch the different parts of the image 460 of the smart mask to initiate the control of the corresponding component of the smart mask. For example, the wearer may touch the air circulation system shown on image 460 to initiate the control function of the air circulation system, corresponding to the icon air control 440.

FIG. 4B illustrates an example air control interface 400B that includes a toggle switch that allows a user to turn on or off the air circulation system. The air control interface 400B also includes a slider that allows the user to control the speed of the fan. In some embodiments, the user can move a bar to any point of the slider 360. In some embodiments, the user can move the bar to a few discrete places, such as at low, medium, high marks.

Note that the user interfaces illustrated in FIGS. 4A and 4B are merely schematic examples. Similar or different user interfaces may be implemented to achieve similar or different functions depending on the model and/or components of the smart mask.

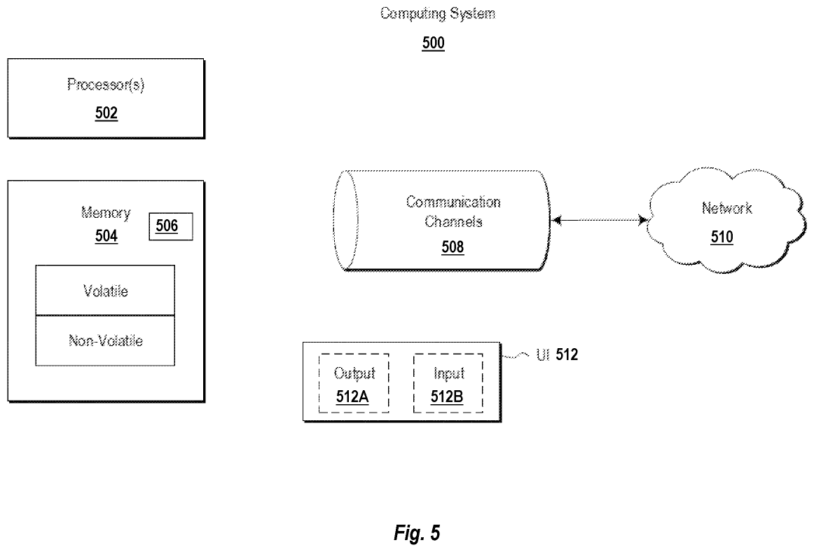

Finally, because the principles described herein may be performed in the context of a computing system (for example, the one or more controller(s) o the smart masks may be a computing system, and the user terminal may be a computing system) some introductory discussion of a computing system will be described with respect to FIG. 5.

Computing systems are now increasingly taking a wide variety of forms. Computing systems may, for example, be handheld devices, appliances, laptop computers, desktop computers, mainframes, distributed computing systems, data centers, or even devices that have not conventionally been considered a computing system, such as wearables (e.g., glasses). In this description and in the claims, the term “computing system” is defined broadly as including any device or system (or a combination thereof) that includes at least one physical and tangible processor, and a physical and tangible memory capable of having thereon computer-executable instructions that may be executed by a processor. The memory may take any form and may depend on the nature and form of the computing system. A computing system may be distributed over a network environment and may include multiple constituent computing systems.

As illustrated in FIG. 5, in its most basic configuration, a computing system 500 typically includes at least one hardware processing unit 502 and memory 504. The processing unit 502 may include a general-purpose processor and may also include a field-programmable gate array (FPGA), an application-specific integrated circuit (ASIC), or any other specialized circuit. The memory 504 may be physical system memory, which may be volatile, non-volatile, or some combination of the two. The term “memory” may also be used herein to refer to non-volatile mass storage, such as physical storage media. If the computing system is distributed, the processing, memory and/or storage capability may be distributed as well.

The computing system 500 also has thereon multiple structures often referred to as an “executable component”. For instance, memory 504 of the computing system 500 is illustrated as including executable component 506. The term “executable component” is the name for a structure that is well understood to one of ordinary skill in the art in the field of computing as being a structure that can be software, hardware, or a combination thereof. For instance, when implemented in software, one of ordinary skill in the art would understand that the structure of an executable component may include software objects, routines, methods, and so forth, that may be executed on the computing system, whether such an executable component exists in the heap of a computing system, or whether the executable component exists on computer-readable storage media.

In such a case, one of ordinary skill in the art will recognize that the structure of the executable component exists on a computer-readable medium such that, when interpreted by one or more processors of a computing system (e.g., by a processor thread), the computing system is caused to perform a function. Such a structure may be computer-readable directly by the processors (as is the case if the executable component were binary). Alternatively, the structure may be structured to be interpretable and/or compiled (whether in a single stage or in multiple stages) so as to generate such binary that is directly interpretable by the processors. Such an understanding of example structures of an executable component is well within the understanding of one of ordinary skill in the art of computing when using the term “executable component”.

The term “executable component” is also well understood by one of ordinary skill as including structures, such as hardcoded or hard-wired logic gates, that are implemented exclusively or near-exclusively in hardware, such as within a field-programmable gate array (FPGA), an application-specific integrated circuit (ASIC), or any other specialized circuit. Accordingly, the term “executable component” is a term for a structure that is well understood by those of ordinary skill in the art of computing, whether implemented in software, hardware, or a combination. In this description, the terms “component”, “agent”, “manager”, “service”, “engine”, “module”, “virtual machine” or the like may also be used. As used in this description and in the case, these terms (whether expressed with or without a modifying clause) are also intended to be synonymous with the term “executable component”, and thus also have a structure that is well understood by those of ordinary skill in the art of computing.

In the description that follows, embodiments are described with reference to acts that are performed by one or more computing systems. If such acts are implemented in software, one or more processors (of the associated computing system that performs the act) direct the operation of the computing system in response to having executed computer-executable instructions that constitute an executable component. For example, such computer-executable instructions may be embodied in one or more computer-readable media that form a computer program product. An example of such an operation involves the manipulation of data. If such acts are implemented exclusively or near-exclusively in hardware, such as within an FPGA or an ASIC, the computer-executable instructions may be hardcoded or hard-wired logic gates. The computer-executable instructions (and the manipulated data) may be stored in the memory 504 of the computing system 500. Computing system 500 may also contain communication channels 508 that allow the computing system 500 to communicate with other computing systems over, for example, network 510.

While not all computing systems require a user interface, in some embodiments, the computing system 500 includes a user interface system 512 for use in interfacing with a user. The user interface system 512 may include output mechanisms 512A as well as input mechanisms 512B. The principles described herein are not limited to the precise output mechanisms 512A or input mechanisms 512B as such will depend on the nature of the device. However, output mechanisms 512A might include, for instance, speakers, displays, tactile output, holograms and so forth. Examples of input mechanisms 512B might include, for instance, microphones, touchscreens, holograms, cameras, keyboards, mouse or other pointer input, sensors of any type, and so forth.

Embodiments described herein may comprise or utilize a special purpose or general-purpose computing system including computer hardware, such as, for example, one or more processors and system memory, as discussed in greater detail below. Embodiments described herein also include physical and other computer-readable media for carrying or storing computer-executable instructions and/or data structures. Such computer-readable media can be any available media that can be accessed by a general-purpose or special purpose computing system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: storage media and transmission media.

Computer-readable storage media includes RAM, ROM, EEPROM, CD-ROM, or other optical disk storage, magnetic disk storage, or other magnetic storage devices, or any other physical and tangible storage medium which can be used to store desired program code means in the form of computer-executable instructions or data structures and which can be accessed by a general-purpose or special purpose computing system.

A “network” is defined as one or more data links that enable the transport of electronic data between computing systems and/or modules and/or other electronic devices. When information is transferred or provided over a network or another communications connection (either hardwired, wireless, or a combination of hardwired or wireless) to a computing system, the computing system properly views the connection as a transmission medium. Transmissions media can include a network and/or data links which can be used to carry desired program code means in the form of computer-executable instructions or data structures and which can be accessed by a general-purpose or special-purpose computing system. Combinations of the above should also be included within the scope of computer-readable media.

Further, upon reaching various computing system components, program code means in the form of computer-executable instructions or data structures can be transferred automatically from transmission media to storage media (or vice versa). For example, computer-executable instructions or data structures received over a network or data link can be buffered in RAM within a network interface module (e.g., a “NIC”), and then eventually transferred to computing system RAM and/or to less volatile storage media at a computing system. Thus, it should be understood that storage media can be included in computing system components that also (or even primarily) utilize transmission media.

Computer-executable instructions comprise, for example, instructions and data which, when executed at a processor, cause a general-purpose computing system, special purpose computing system, or special purpose processing device to perform a certain function or group of functions. Alternatively, or in addition, the computer-executable instructions may configure the computing system to perform a certain function or group of functions. The computer executable instructions may be, for example, binaries or even instructions that undergo some translation (such as compilation) before direct execution by the processors, such as intermediate format instructions such as assembly language, or even source code.

Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, data centers, wearables (such as glasses) and the like. The invention may also be practiced in distributed system environments where local and remote computing system, which are linked (either by hardwired data links, wireless data links, or by a combination of hardwired and wireless data links) through a network, both perform tasks. In a distributed system environment, program modules may be located in both local and remote memory storage devices.

Those skilled in the art will also appreciate that the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

The remaining figures may discuss various computing system which may correspond to the computing system 500 previously described. The computing systems of the remaining figures include various components or functional blocks that may implement the various embodiments disclosed herein as will be explained. The various components or functional blocks may be implemented on a local computing system or may be implemented on a distributed computing system that includes elements resident in the cloud or that implement aspect of cloud computing. The various components or functional blocks may be implemented as software, hardware, or a combination of software and hardware. The computing systems of the remaining figures may include more or less than the components illustrated in the figures and some of the components may be combined as circumstances warrant. Although not necessarily illustrated, the various components of the computing systems may access and/or utilize a processor and memory, such as processor 502 and memory 504, as needed to perform their various functions.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A smart mask, comprising:
a face-covering configured to cover a face area of a wearer;
an attachment element configured to attach the face-covering onto the face area of the wearer;
one or more power source(s);
an air circulation system powered by at least one of the one or more power source(s) and configured to filter outside air and draw the filtered outside air into the face area;
an exhaust system placed below the air circulation system and configured to purge exhaled air out of the face area; and
a controller configured to control the air circulation system.

2. The smart mask of claim 1, wherein the air circulation system comprises at least:
one or more filter receptacle(s), each of which is configured to receive a filter cartridge for filtering the outside air drawn into the face area;
one or more fan(s), each of which is powered by at least one of the one or more power source(s) and configured to draw outside air through a corresponding filter cartridge into the face area; and
one or more puck(s), each of which is configured to removably cover the corresponding filter cartridge or fan.

3. The smart mask of claim 2, wherein the one or more fan(s) include a left fan placed on a left side of the face area and a right fan placed on a right side of the face area;
the one or more filter receptacle(s) includes (1) a left filter receptacle coupled to the left fan and configured to receive a left filter cartridge, and (2) a right filter receptacle coupled to the right fan and configured to receive a right filter cartridge; and
the one or more puck(s) includes a left puck configured to cover the left fan and the left filter cartridge, and a right puck configured to cover the right fan and the right filter cartridge.

4. The smart mask of claim 2, wherein each of the one or more filter receptacle(s) comprises a magnetic portion, configured to hold a metal frame of the filter cartridge in place magnetically.

5. The smart mask of claim 2, wherein each of the one or more filter receptacle(s) has a circular-shaped recess configured to receive a circular-shaped filter cartridge.

6. The smart of claim 2, wherein each of the one or more fan(s) is configured to operate at different speeds, and the controller is configured to control a speed of the one or more fan(s).

7. The smart mask of claim 2, further comprising:
one or more ultraviolet light sources powered by at least one of the one or more power source(s) and configured to disinfect an area around the filter cartridge, wherein the controller is further configured control the one or more ultraviolet light source(s).

8. The smart mask of claim 7, wherein each of the one or more ultraviolet light source(s) is coupled to a corresponding one of the one or more puck(s) or a corresponding one of the one or more filter receptacle(s).

9. The smart mask of claim 8, wherein each of the one or more ultraviolet light source(s) is configured to emit an ultraviolet light at different intensities, and the controller is further configured to control an intensity of the one or more ultraviolet light source(s).

10. The smart mask of claim 1, further comprising:
an audio system powered by at least one of the one or more power source(s) and configured to receive a sound signal wirelessly from a user terminal; and
the controller is further configured to control the audio system.

11. The smart mask of claim 10, wherein the audio system includes at least (1) a pair of earbuds or (2) a pair of speakers.

12. The smart mask of claim 11, wherein each of the pair of earbuds includes a magnetic portion or a metal portion,
the attachment element also includes a pair of magnetic portions or metal portions corresponding to the pair of earbuds, such that when the pair of earbuds are not in use, each of the pair of earbuds is configured to be secured to the corresponding magnetic portion or metal portion of the attachment element magnetically.

13. The smart mask of claim 10, wherein the audio system further includes a microphone configured to receive a voice input, and the controller is further configured to process the voice input to identify one or more voice command(s) for controlling the air circulation system or the audio system.

14. The smart mask of claim 1, further comprising:
a network interface configured to connect to a user terminal, wherein when the user terminal is connected to the network interface, the network interface is configured to transmit status data to the user terminal, and to grant the user terminal control of the air circulation system.

15. The smart mask of claim 1, further comprising:
a temperature sensor configured to detect a body temperature of the wearer.

16. The smart mask of claim 1, further comprising:
a heart rate monitor configured to detect a heart rate of the wearer.

17. The smart mask of claim 1, wherein the one or more power source(s) include one or more rechargeable batteries, and the smart mask further comprises a charging port configured to charge the one or more rechargeable batteries.

18. A filter cartridge configured to fit a face mask having a fan and a receptacle for receiving a filter, comprising:
a plurality of layers of fabric; and
a metal frame configured to snap onto a magnetic portion of the receptacle of the face mask.

19. The filter cartridge of claim 18, further comprising:
an aperture in a center of the plurality of layers of fabric to allow a nose of the fan of the face mask to protrude through.

20. A smart mask, comprising:
a face-covering configured to cover a face area of a wearer;
an attachment element configured to attach the face-covering onto the face of the wearer;
one or more power source(s);
an air circulation system powered by at least one of the one or more power source(s) and configured to filter outside air and draw filtered outside air into the face area;
one or more ultraviolet light source(s) powered by at least one of the one or more power source(s) and configured to disinfect an area of the face-covering around the wearer's mouth;
an exhaust system configured to purge exhaled air out of the face area; and
a controller configured to control the air circulation system and the one or more ultraviolet light source(s).
